(19) United States
(12) Patent Application Publication
(10) Pub. No.: US 2024/0032781 A1
(43) Pub. Date: Feb. 1, 2024

(54) IMAGE PICKUP UNIT AND ENDOSCOPE

(71) Applicant: OLYMPUS MEDICAL SYSTEMS CORP., Tokyo (JP)

(72) Inventors: Yo ASAKURA, Tokyo (JP); Takuro HORIBE, Funabashi-shi (JP)

(73) Assignee: OLYMPUS MEDICAL SYSTEMS CORP., Tokyo (JP)

(21) Appl. No.: 18/378,839

(22) Filed: Oct. 11, 2023

Related U.S. Application Data
(63) Continuation of application No. PCT/JP2021/021811, filed on Jun. 8, 2021.

(51) Int. Cl. A61B 1/05; A61B 1/00

(52) U.S. Cl. CPC A61B 1/051; A61B 1/0011

(57) ABSTRACT

An image pickup unit includes a three-dimensional substrate which includes a recess portion and an image pickup module which is installed in the recess portion, and the recess portion includes a first recess portion, a second recess portion which communicates with the first recess portion and which has a wider opening and a shallower depth than the first recess portion, and a third recess portion which is formed by providing a notch from a bottom surface of the second recess portion in a depth direction of the first recess portion.

### CROSS REFERENCE TO RELATED APPLICATION

This application is a continuation application of PCT/JP2021/021811 filed on Jun. 8, 2021, the entire contents of which are incorporated herein by this reference.

### BACKGROUND OF THE INVENTION

### 1. Field of the Invention

The present invention relates to an image pickup unit provided with an image pickup module and an endoscope including the image pickup unit.

### 2. Description of the Related Art

In related art, in a medical field, an endoscope has widely been used which can observe organs and so forth in a body cavity by inserting an elongated insertion portion into the body cavity. In a case where an observation image of the organs and so forth in the body cavity is displayed on a monitor, an endoscope or the like is used in which an image sensor having a charge coupled device (CCD or CMOS) or the like is disposed in an image pickup portion at a distal end or a rear end of an insertion portion of the endoscope.

In recent years, as for an endoscope, reduction in diameter of an insertion portion has been requested for minimally invasive examination and treatment, and size reduction of a distal end portion of the insertion portion has also been requested. Thus, there is a case where a wafer-level camera is used as a small-sized camera module for an image pickup unit mounted on the distal end portion of the insertion portion.

In regards to techniques for mounting an electronic component such as a small-sized camera module on a substrate, for example, Japanese Patent Application Laid-Open Publication No. 2014-157864 discloses a stacked substrate in which an alignment mark is provided to a bottom surface of a recess portion on which the electronic component is mounted and positioning precision is thereby improved.

### SUMMARY OF THE INVENTION

An image pickup unit in one aspect of the present invention includes: a three-dimensional substrate which includes a recess portion; and an image pickup module which is installed in the recess portion, and the recess portion includes a first recess portion, a second recess portion which communicates with the first recess portion and which has a wider opening and a shallower depth than the first recess portion, and a third recess portion which is formed by providing a notch from a bottom surface of the second recess portion in a depth direction of the first recess portion.

An endoscope in one aspect of the present invention includes: a three-dimensional substrate which includes a recess portion; and an image pickup module which is installed in the recess portion, and the recess portion includes a first recess portion, a second recess portion which communicates with the first recess portion and which has a wider opening and a shallower depth than the first recess portion, and a third recess portion which is formed by providing a notch from a bottom surface of the second recess portion in a depth direction of the first recess portion.

### BRIEF DESCRIPTION OF THE DRAWINGS

FIG. 1 is a side view illustrating a configuration of an endoscope in one form of the present invention;

FIG. 2 is a partial cross-sectional view illustrating a configuration of a distal end portion at time before a distal end cover is attached, in the form of the present invention;

FIG. 3 is a top view illustrating the configuration of the distal end portion, in which the distal end cover is attached, in the form of the present invention;

FIG. 4 is a perspective view illustrating a configuration of an image pickup unit in the form of the present invention;

FIG. 5 is a top view illustrating the configuration of the image pickup unit at a time before resin sealing, in the form of the present invention;

FIG. 6 is a cross-sectional view illustrating, along line VI-VI in FIG. 5, the configuration of the image pickup unit at a time before the resin sealing, in the form of the present invention;

FIG. 7 is a top view illustrating the configuration of the image pickup unit at a time after the resin sealing, in the form of the present invention;

FIG. 8 is a cross-sectional view illustrating, along line VIII-VIII in FIG. 7, the configuration of the image pickup unit at a time after the resin sealing, in the form of the present invention;

FIG. 9 is a perspective view illustrating a configuration of an image pickup unit at a time before the resin sealing, in a first modification;

FIG. 10 is a top view illustrating the configuration of the image pickup unit at a time before the resin sealing, in the first modification;

FIG. 11 is a cross-sectional view illustrating the configuration of the image pickup unit at a time before the resin sealing, in the first modification;

FIG. 12 is a top view illustrating the configuration of the image pickup unit at a time after the resin sealing, in the first modification;

FIG. 13 is a cross-sectional view illustrating the configuration of the image pickup unit at a time after the resin sealing, in the first modification;

FIG. 14 is a perspective view illustrating a configuration of an image pickup unit at a time before the resin sealing, in a second modification;

FIG. 15 is a top view illustrating the configuration of the image pickup unit at a time before the resin sealing, in the second modification; and

FIG. 16 is a perspective view illustrating a configuration of an image pickup unit at a time before the resin sealing, in a third modification.

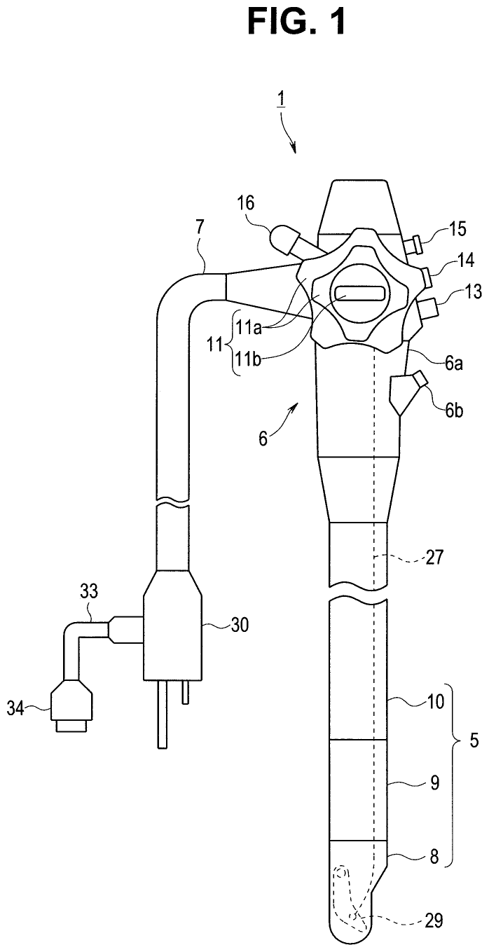

### DETAILED DESCRIPTION OF THE PREFERRED EMBODIMENTS

A description will herein be made by raising forms of an endoscope and an image pickup unit as examples. Note that in the following descriptions, drawings based on each embodiment are schematic drawings, attention has to be paid to a fact that a relationship between a thickness and a width of each portion, ratios among thicknesses of respective portions, and so forth are different from reality, and the drawings may include portions about which dimensional relationships and ratios are mutually different among the drawings.

An endoscope in the following configuration descriptions will be described by raising, as an example, a so-called flexible scope which has flexibility in an insertion portion for insertion into a digestive organ or the like in an upper portion or a lower portion of a living body. However, this is not restrictive, and a technique can be applied to a so-called rigid scope for surgical use in which an insertion portion inserted into an abdominal cavity or the like of a living body is rigid. The technique is not limited to a medical endoscope but can be applied to an industrial endoscope.

A configuration of the image pickup unit is not limited to an image pickup unit mounted on an endoscope, but the technique can be applied to image pickup units mounted on various apparatuses.

### Embodiment

An endoscope in one form of the present invention will hereinafter be described based on the drawings.

An endoscope 1 as an insertion apparatus of the present embodiment has an insertion portion 5 inserted into a body, an operation portion 6, and a universal cable 7. The insertion portion 5 is an elongated long-length member which is inserted into an observation target part from a distal end side in a longitudinal axis direction. The insertion portion 5 is configured with a distal end portion 8, a bending portion 9, and a flexible tube portion 10 which are connectedly provided.

An illumination optical system including a light guide and an image pickup optical system including an image pickup device are built in the distal end portion 8, and a nozzle and a suction opening, which is also used as a treatment instrument guide-out opening, are provided to a distal end surface (none is illustrated here).

An observation window and an illumination window which have predetermined angles with respect to an insertion direction of the insertion portion 5 are provided to the distal end portion 8. A raising base (forceps elevator) 29 which is a movable portion as a direction switching portion which raises a treatment instrument and switches a direction of the treatment instrument to an observation direction is disposed in the distal end portion 8.

The raising base 29 is connected to a raising base operation wire (hereinafter, simply abbreviated as wire) 27 as a pulling-loosening member which is a long-length member inserted through insides of the insertion portion 5 and the operation portion 6. The wire 27 is pulled and loosened, and a raising-lowering operation for the raising base 29 is thereby performed. Note that the wire 27 is pulled and loosened by an operation of a raising base operation lever 16.

The bending portion 9 is configured to be bendable in four directions of up, down, left, and right, for example. The flexible tube portion 10 is a tube-shaped member which has a long length and flexibility.

The operation portion 6 includes a grasping portion 6a. The grasping portion 6a is connectedly provided to a proximal end portion of the insertion portion 5. A treatment instrument insertion opening 6b is disposed in the operation portion 6.

A bending operation portion 11, an air/water feeding button 13, a suction button 14, a cleaning tube mounting pipe sleeve 15, and so forth are provided to the operation portion 6. The raising base operation lever 16 as an operation member is provided to the operation portion 6.

The bending operation portion 11 has a bending operation knob 11a and a fixing lever 11b. The bending operation knob 11a is an operation portion for performing a bending operation of the bending portion 9 of the insertion portion 5. The fixing lever 11b is an operation portion for fixing the bending operation knob 11a to a desired rotation position.

The universal cable 7 extends from a side surface of the operation portion 6. An endoscope connector 30 which is connected to a light source device as an external apparatus is provided to an end portion of the universal cable 7. A signal transmission cable 33 is provided to extend from a side portion of the endoscope connector 30. An electrical connector 34 which is connected to a video processor is provided to another end side of the signal transmission cable 33.

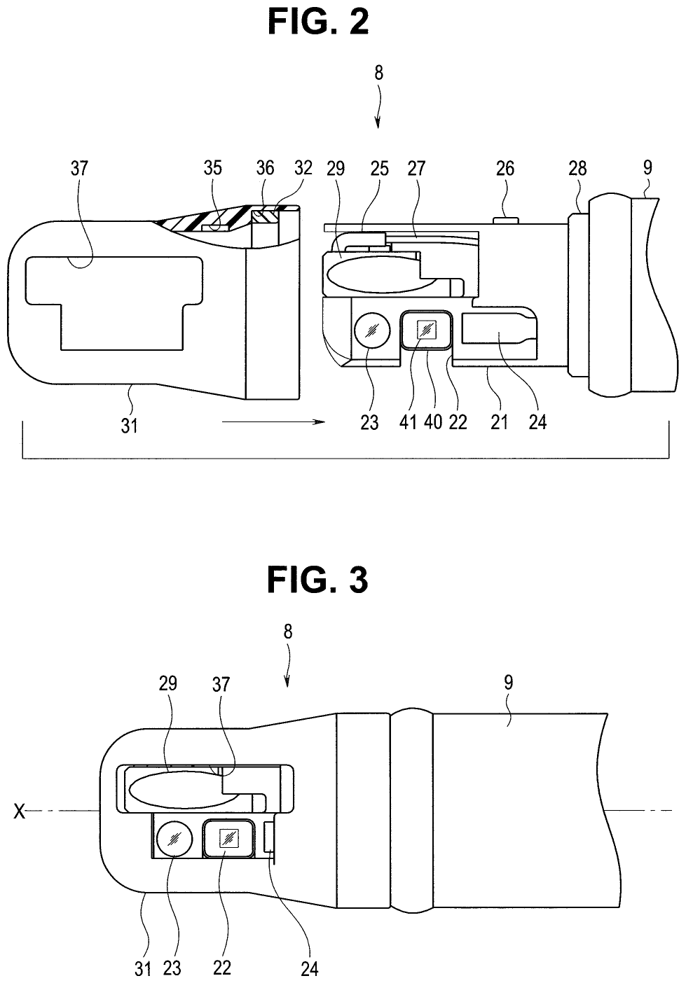

As illustrated in FIG. 2, a distal end configuring portion 21 formed of metal such as stainless steel is provided to the distal end portion 8 of the insertion portion 5. The raising base 29 formed of metal such as stainless steel is rotatably disposed in the distal end configuring portion 21.

In the distal end configuring portion 21, an observation window 41 and an illumination window 23 are provided to one side surface orthogonal to an insertion axis X illustrated in FIG. 3. A nozzle 24 for feeding air and water toward the observation window 41 and the illumination window 23 is provided to the distal end configuring portion 21.

An insulation ring 28 as a first insulation member is provided to a proximal end outer peripheral portion of the distal end configuring portion 21. Note that the insulation ring 28 is formed of non-metal such as electrically non-conductive resin or ceramics.

The raising base 29 is rotatably provided and is pivotally supported in the distal end configuring portion 21 by a rotating shaft. The raising base 29 is connected to one side surface by a connecting body 25 which is formed into an L shape by crooking the wire 27. The raising base 29 is raised and lowered around the rotating shaft by a pulling-loosening operation of the wire 27.

A distal end cover 31 as a cover body is detachably attached to the distal end configuring portion 21. The distal end cover 31 is attached to cover the distal end configuring portion 21. Note that the distal end cover 31 is formed of synthetic resin such as an elastic-plastic material having electric insulation.

A locking recess portion 35 engages with a protrusion-portion-shaped locking pin 26, which is illustrated in FIG. 2, as a locking portion which is provided to project from a side portion of the distal end configuring portion 21, and the distal end cover 31 is thereby attached and fixed to the distal end configuring portion 21 as illustrated in FIG. 3. Note that the locking recess portion 35 is a locked portion which is formed in an inner peripheral portion of the distal end cover 31.

The distal end cover 31 has an opening portion 37 for exposing the observation window 41 and the illumination window 23 and for guiding out the treatment instrument or the like which is raised and lowered by the raising base 29.

In the distal end cover 31, a step portion 36 is formed in a circumferential direction of a proximal end internal portion. In the distal end cover 31, an elastic and ring-shaped rubber cover 32 having electric insulation is joined to the step portion 36.

In other words, the rubber cover 32 as an insulation member is disposed on a whole periphery of a proximal end inner peripheral portion of the distal end cover 31. The rubber cover 32 is formed of rubber or the like which is more flexible than the distal end cover 31. Accordingly, in a state where the distal end cover 31 is attached to the distal end configuring portion 21, a proximal end portion of the distal end cover 31 covers the insulation ring 28 provided to a proximal end outer periphery of the distal end configuring portion 21, and a whole periphery of a proximal end surface of the rubber cover 32 tightly fits a whole periphery of a distal end surface of the insulation ring 28. In this case, the rubber cover 62 tightly fits a bonding portion to the insulation ring 28 and is thereby set to a state where watertightness is retained.

An image pickup unit 40 including the observation window 41 is mounted on the distal end configuring portion 21. The image pickup unit 40 is disposed in a recess-portion-shaped unit mounting chamber 22 which is formed by notching the side portion of the distal end configuring portion 21.

Here, a detailed description will be made about a configuration of the image pickup unit 40 of the present embodiment.

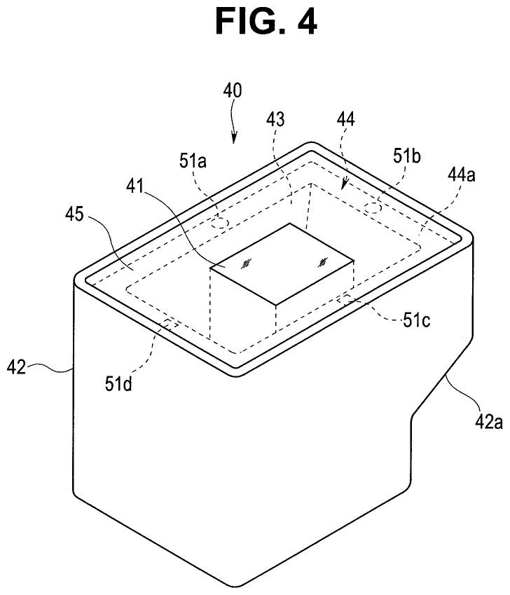

As illustrated in FIG. 4, the image pickup unit 40 has a main external shape by a block-shaped three-dimensional substrate 42 in which one surface (upper surface) on an exposed side in the distal end portion 8 has a rectangular shape and an inclined surface 42a is formed on a proximal end side of a halfway portion on the other surface side (lower surface side).

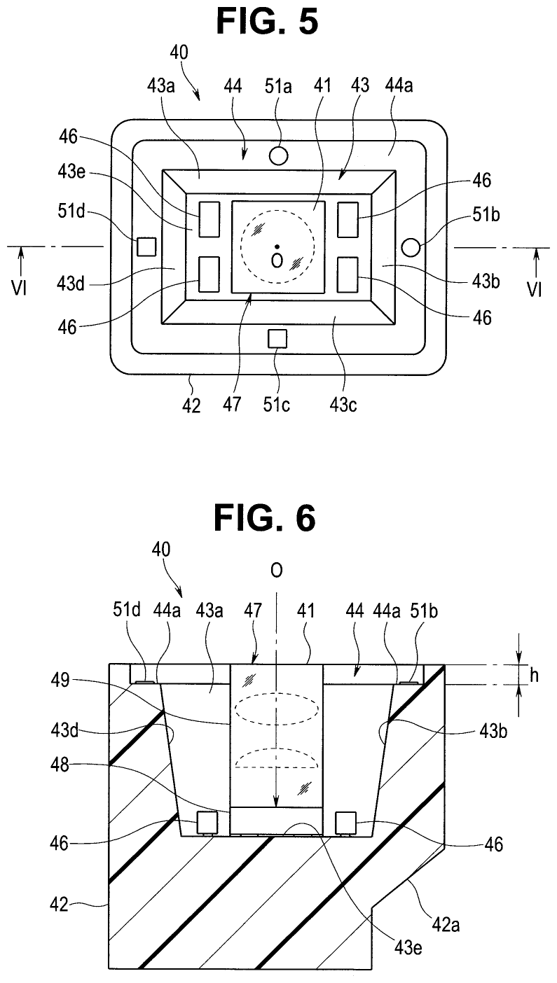

As illustrated in FIG. 5 and FIG. 6, an electronic component placement recess portion 43 as a first recess portion is formed in the three-dimensional substrate 42. In the electronic component placement recess portion 43, here, four electronic components 46 such as a camera module 47 as a wafer-level camera and a chip capacitor are mounted on a bottom surface 43e. The three-dimensional substrate 42 has an alignment placement recess portion 44 as a second recess portion which has a larger opening than the electronic component placement recess portion 43.

More specifically, the electronic component placement recess portion 43 has a predetermined depth which is generally the same as a height of the camera module 47. In the electronic component placement recess portion 43, four wall surfaces 43a, 43b, 43c, and 43d here are formed with inclined surfaces which have predetermined angles toward a deep portion of the three-dimensional substrate 42. In other words, the electronic component placement recess portion 43 forms a space shape of an inverted quadrangular frustum.

Note that here, the three-dimensional substrate 42 is an MID (molded interconnect device) of a resin molded component in which wiring, electrodes, and so forth are formed, for example.

The alignment placement recess portion 44 has a predetermined depth h in a depth direction of the three-dimensional substrate 42 along edge side portions of one surface (upper surface) of the three-dimensional substrate 42. The alignment placement recess portion 44 has flat surfaces 44a having predetermined widths to an opening of the electronic component placement recess portion 43. In other words, in the alignment placement recess portion 44, a step in the depth direction of the three-dimensional substrate 42 is formed in a portion from the edge side portions of the three-dimensional substrate 42 to the opening of the electronic component placement recess portion 43. The alignment placement recess portion 44 has the flat surfaces 44a in rectangular belt shapes along four edge side portions of the three-dimensional substrate 42. The four flat surfaces 44a provided along the edge side portions are coupled together in a frame shape.

On the belt-shaped flat surfaces 44a of the alignment placement recess portion 44, here, for example, a total of four alignment marks 51a, 51b, 51c, and 51d which are circular or quadrangular are printed at general centers of four sides by plating or the like.

Note that four belt-shaped flat surfaces 44a on which the alignment marks 51a, 51b, 51c, and 51d are provided are provided to a whole periphery along the four edge side portions of the three-dimensional substrate 42. However, this is not restrictive, and one or more flat surfaces may be provided in any of the edge side portions of the three-dimensional substrate 42. The flat surface 44a in the rectangular belt shape may have another shape than the rectangular belt shape and may be formed into a surface which is other than a flat surface and has a bending surface or a step.

The camera module 47 includes an image sensor 48 and a lens assembly 49, and an external shape of the camera module 47 has a quadrangular prism shape in which the observation window 41 serves as a surface. The image sensor 48 is a CCD or a CMOS which performs photoelectric conversion of image pickup light of an optical axis O. The lens assembly 49 is an optical system which collects the image pickup light of the optical axis O.

The camera module 47 is installed in a terminal, which is not illustrated, of the bottom surface 43e of the electronic component placement recess portion 43 of the three-dimensional substrate 42 by reflow soldering while the alignment marks 51a, 51b, 51c, and 51d provided to the flat surfaces 44a of the alignment placement recess portion 44 are checked.

Note that each of the electronic components 46 such as the chip capacitor is also installed in a terminal (not illustrated) of the bottom surface 43e of the electronic component placement recess portion 43 of the three-dimensional substrate 42 by reflow soldering while the alignment marks 51a, 51b, 51c, and 51d are checked.

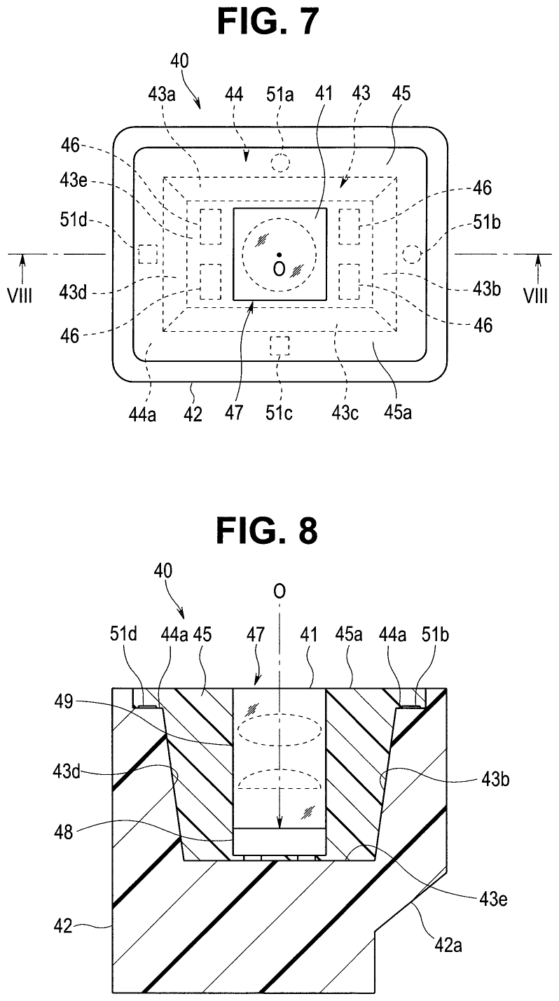

As illustrated in FIG. 7 and FIG. 8, as for the image pickup unit 40, after the camera module 47 and each of the electronic components 46 are installed in the electronic component placement recess portion 43 of the three-dimensional substrate 42, the electronic component placement recess portion 43 and the alignment placement recess portion 44 are filled with a light-blocking underfill material 45 such as rigid resin, and a heat curing process is performed.

In this case, filling with the underfill material 45 is performed such that the underfill material 45 is in the same plane as a surface of the observation window 41. In other words, filling with the underfill material 45 is performed such that the surface of the observation window 41 is exposed and the surface of the observation window 41 and a surface 45a (see FIG. 8) of the underfill material 45 are in the same plane.

Note that surfaces of the edge side portions of the three-dimensional substrate 42 are also in the same plane as the surface of the observation window 41 and the surface 45a of the underfill material 45.

When the filling with the underfill material 45 is performed, a part for resin filling by a resin filling nozzle (not illustrated) is positioned while the alignment marks 51a, 51b, 51c, and 51d are also used as filling points.

In such a manner, in the image pickup unit 40, the electronic component placement recess portion 43 as the first recess portion is provided to the three-dimensional substrate 42. In the image pickup unit 40, the alignment placement recess portion 44 as the second recess portion, the opening of which is wider than the electronic component placement recess portion 43 and the depth of which is shallower than the electronic component placement recess portion 43, is formed on a surface side of the three-dimensional substrate 42 while the alignment placement recess portion 44 communicates (is connected) with the opening of the electronic component placement recess portion 43.

In the image pickup unit 40, the camera module 47 as an image pickup module and each of the electronic components 46 are installed while the alignment marks 51a, 51b, 51c, and 51d provided to the belt-shaped flat surfaces 44a serving as bottom surfaces of the alignment placement recess portion 44 are used as indicators of mounting positions. Note that the camera module 47 as the image pickup module is installed in the bottom surface 43e of the electronic component placement recess portion 43.

Next, the electronic component placement recess portion 43 and the alignment placement recess portion 44 are filled with the underfill material 45 which has light blocking characteristics such that the observation window 41 of the camera module 47 is exposed. In this case also, the alignment marks 51a, 51b, 51c, and 51d are used for positioning for the resin filling by the resin filling nozzle (not illustrated).

In the image pickup unit 40, in a case where filling resin goes over the electronic component placement recess portion 43 and moves to the alignment placement recess portion 44 when the filling with the underfill material 45 is performed, a situation where an area of the resin expands can be checked. Thus, the image pickup unit 40 can prevent the filling resin from overflowing from the edge side portions (surface) of the three-dimensional substrate 42.

Note that in the image pickup unit 40, biocompatible resin is used for the underfill material 45. The image pickup unit 40 has a configuration in which the alignment marks 51a, 51b, 51c, and 51d are sealed by the underfill material 45 and are not exposed on the surface of the three-dimensional substrate 42.

As described above, the image pickup unit 40 installed in the distal end portion 8 of the insertion portion 5 of the endoscope 1 can have a configuration in which even when spaces between the camera module 47 and the electronic components 46 are made small with respect to the wall surfaces 43a, 43b, 43c, and 43d of the electronic component placement recess portion 43, the alignment marks 51a, 51b, 51c, and 51d provided to the belt-shaped flat surfaces 44a serving as the bottom surfaces of the alignment placement recess portion 44 do not disappear but are easily read.

Thus, the image pickup unit 40 has a configuration which can achieve size reduction without hindering reading of the alignment marks 51a, 51b, 51c, and 51d. The endoscope 1 in which the image pickup unit 40 is mounted on the distal end portion 8 of the insertion portion 5 can achieve size reduction of the distal end portion 8 and reduction in diameter of the insertion portion 5.

### (First Modification)

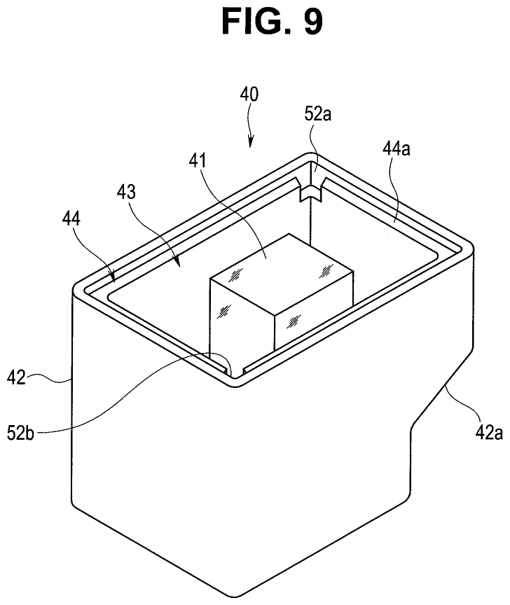

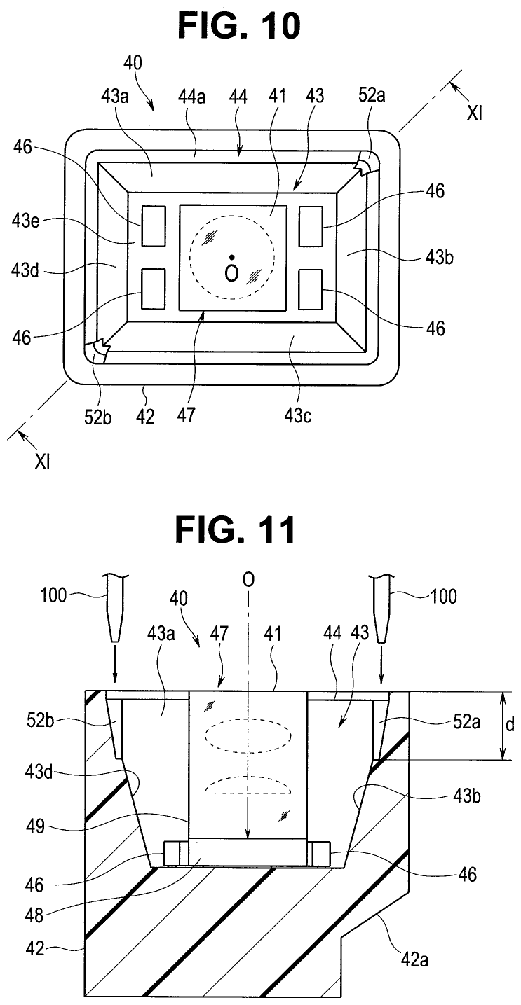

As illustrated in FIG. 9 and FIG. 10, in the image pickup unit 40 of the present modification, here, two notches 52a and 52b which are also used as alignment marks as third recess portions are formed in the flat surfaces 44a, in the rectangular belt shapes, of the alignment placement recess portion 44 as the second recess portion of the three-dimensional substrate 42.

The two notches 52a and 52b are formed in corner portions of the alignment placement recess portion 44 of the three-dimensional substrate 42. Note that the two notches 52a and 52b are provided to the corner portions in symmetrical positions with respect to the camera module 47.

In other words, the two notches 52a and 52b are formed in corner portions in point-symmetrical positions in the three-dimensional substrate 42. Accordingly, stress, thermal stress, and so forth which are exerted on the camera module 47 can be made uniform.

Note that as illustrated in FIG. 11, the two notches 52a and 52b are formed from the observation window 41 as the surface of the camera module 47 to a predetermined depth d in the depth direction of the three-dimensional substrate 42.

For example, the predetermined depth d of the notches 52a and 52b from the observation window 41 is set in a range of 0 mm<d<0.15 mm. In other words, bottom surfaces of the notches 52a and 52b are provided in low positions which are deeper than 0 mm and less deep than 0.15 mm from the observation window 41.

In such a configuration, in the image pickup unit 40, the camera module 47 as the image pickup module and each of the electronic components 46 are installed in the bottom surface 43e of the electronic component placement recess portion 43 while the two notches 52a and 52b as alignment marks are checked.

Subsequently, the two notches 52a and 52b as the third recess portions also function as alignment marks for checking positions of resin application nozzles 100 for performing filling with the underfill material 45.

In this case, the resin application nozzles 100 are deeply inserted into the two notches 52a and 52b, and the electronic component placement recess portion 43 and the alignment placement recess portion 44 of the three-dimensional substrate 42 are filled with the light-blocking underfill material 45 such as rigid resin.

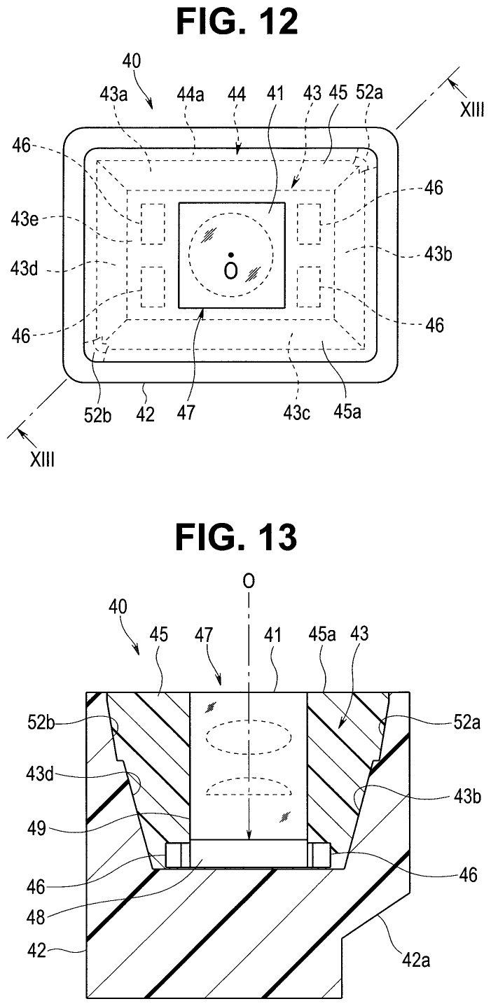

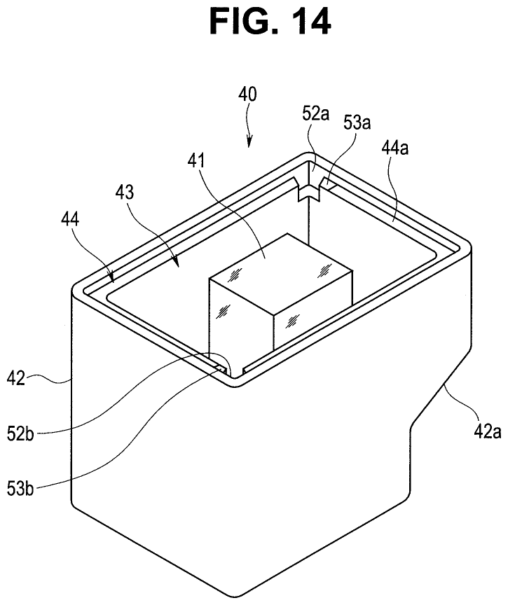

Accordingly, as illustrated in FIG. 12 and FIG. 13, the image pickup unit 40 is filled with the underfill material 45 such that the surface of the observation window 41 is exposed and the underfill material 45 is in the same plane as the surface of the observation window 41.

Note that the two notches 52a and 52b are formed in the three-dimensional substrate 42 of the image pickup unit 40, and it thereby becomes possible to insert the large-diameter resin application nozzles 100 having large inner diameters into the two notches 52a and 52b. Accordingly, the image pickup unit 40 can improve efficiency of filling work with the underfill material 45.

In the present modification, a description is made about a configuration in which the two notches 52a and 52b are provided to the corner portions of the three-dimensional substrate 42 in symmetrical positions with respect to the camera module 47. However, this is not restrictive, and four notches may be formed in all corner portions of the three-dimensional substrate 42.

As long as positions are symmetrical positions with respect to the camera module 47, positions in which plural notches 52a and 52b are provided may not be corner portions of the three-dimensional substrate 42.

### (Second Modification)

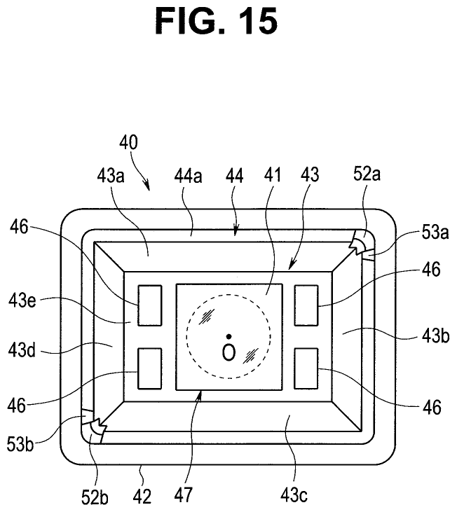

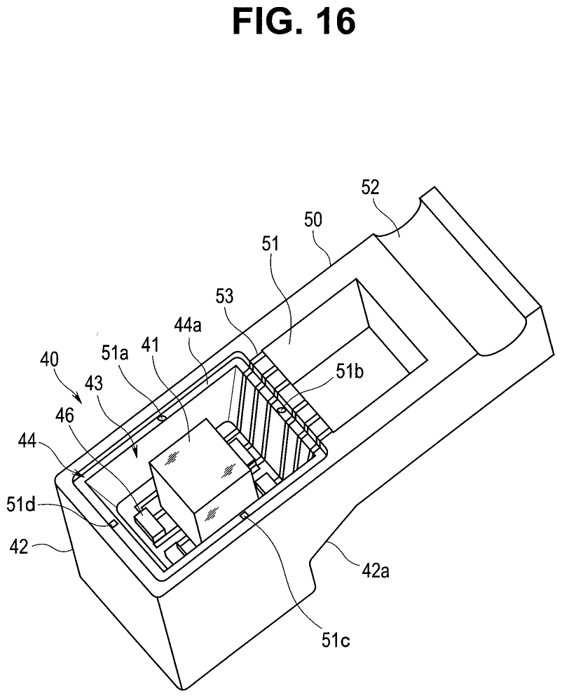

Note that as illustrated in FIG. 14 and FIG. 15, alignment marks 53a and 53b are further provided to the flat surfaces 44a which are adjacent to the two notches 52a and 52b as the third recess portions and which serve as the bottom surfaces of the alignment placement recess portion 44 as the second recess portion, and visibility can thereby further be improved.

### (Third Modification)

As illustrated in FIG. 16, the image pickup unit 40 of the present modification has a configuration in which a base portion 50 provided to extend on a proximal end side is integrally formed with the three-dimensional substrate 42 to which the alignment marks 51a, 51b, 51c, and 51d are provided.

In the image pickup unit 40, a rectangular hole portion 51 is formed in a halfway portion of the base portion 50, and the image pickup unit 40 has a combining portion 52 in a proximal end portion of the base portion 50 in order to enable the image pickup unit 40 to be easily combined with the distal end portion 8 of the endoscope 1.

The combining portion 52 has a recess portion shape such that positioning can be performed by using a screw or the like, for example. In other words, in the image pickup unit 40, the base portion 50 as an extension portion in the three-dimensional substrate 42 is caused to correspond to the insertion direction of the insertion portion 5, for example, and a dimension in a radial direction, which may obstruct insertion, is thereby maintained small.

The base portion 50 is provided to the three-dimensional substrate 42 in such a manner, and the base portion 50 side can thereby be dealt with such that a defect such as a flaw does not occur due to contact with a metal wiring pattern 53 in combining work of the image pickup unit 40 with the distal end portion 8. Thus, a configuration becomes possible in which handling in manufacturing products or in examining the image pickup unit 40 is improved.

Configurations of each of the embodiment and the modifications, which are described above, may be combined with each other, and in addition, it is possible to carry out various modifications in phases for carrying out embodiments without departing from the scope of the gist of the present invention. The above embodiment includes inventions in various phases, and various inventions can be extracted by appropriate combinations of plural disclosed configuration elements.

For example, even when several configuration elements are erased from all of the configuration elements described in the embodiment, in a case where described problems can be solved and described effects can be obtained, configurations from which the configuration elements are erased can be extracted as inventions.

What is claimed is:

1. An image pickup unit comprising:
a three-dimensional substrate which includes a recess portion; and
an image pickup module which is installed in the recess portion, wherein
the recess portion includes a first recess portion, a second recess portion which communicates with the first recess portion and which has a wider opening and a shallower depth than the first recess portion, and a third recess portion which is formed by providing a notch from a bottom surface of the second recess portion in a depth direction of the first recess portion.

2. The image pickup unit according to claim 1, wherein
an alignment mark is provided to the bottom surface of the second recess portion,
the first recess portion and the second recess portion are filled with resin, and
the alignment mark is sealed by the resin.

3. The image pickup unit according to claim 1, wherein
the third recess portion has a dimension in which a resin application nozzle is inserted.

4. The image pickup unit according to claim 1, wherein
an alignment mark is provided to the bottom surface, which is adjacent to the third recess portion, of the second recess portion.

5. The image pickup unit according to claim 3, wherein
the third recess portion in plurality is provided in symmetrical positions with respect to the image pickup module.

6. The image pickup unit according to claim 1, wherein
an observation window of the image pickup module is positioned on a surface side of the three-dimensional substrate relative to the bottom surface of the second recess portion.

7. The image pickup unit according to claim 1, wherein
the bottom surface is formed into a flat surface in a rectangular belt shape.

8. The image pickup unit according to claim 7, wherein
the three-dimensional substrate includes an edge side portion, and
the bottom surface is provided along the edge side portion.

9. The image pickup unit according to claim 8, wherein
the three-dimensional substrate includes the edge side portion, the number of which is four, and the bottom surface, the number of which is four, and
the four bottom surfaces are coupled in a frame shape.

10. An endoscope comprising:
a three-dimensional substrate which includes a recess portion;
an image pickup module which is installed in the recess portion; and
an image pickup unit in which the recess portion includes a first recess portion, a second recess portion which communicates with the first recess portion and which has a wider opening and a shallower depth than the first recess portion, and a third recess portion which is formed by providing a notch from a bottom surface of the second recess portion in a depth direction of the first recess portion, wherein
the image pickup unit is installed in a distal end portion of an insertion portion.
